(19) United States
(12) Patent Application Publication
(10) Pub. No.: US 2010/0129371 A1
(43) Pub. Date: May 27, 2010

(54) Method for genetic immunization by electrotransfer against a toxin and antiserum obtainable by said method

(75) Inventors: Daniel Scherman, Paris (FR); Pascal Bigey, Paris (FR); Capucine Trollet, Paris (FR); Michel R. Popoff, Clamart (FR); Yannick Pereira, Paris (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignees: Centre National de la Recherche Scientifique (CNRS), Paris (FR); Institute Pasteur, Paris (FR); Institute National de la Sante et de la Recherche Medicale (INSERM), Paris (FR); Universite Rene Descartes, Paris (FR)

(21) Appl. No.: 11/989,573

(22) PCT Filed: Jul. 28, 2006
(86) PCT No.: PCT/EP2006/064798

§ 371 Date: Sep. 15, 2009

(30) Foreign Application Priority Data
Jul. 28, 2005 (FR) 0508065

(51) Int. Cl. A61K 39/395; A61P 37/04

(52) U.S. Cl. 424/139.1; 424/130.1

(57) ABSTRACT

The invention concerns a method for obtaining an antiserum directed against a proteinic toxin by administering to an animal a solution comprising a genetic construct encoding a toxin immunogenic fragment, then applying an electric field in the administering zone, and isolating the serum. The invention also concerns the antiserum obtainable by the method as well as the use of the solution for making a medicine for preventing or treating a toxic effect related to absorption by a mammal of a toxin. The invention is characterized in that said medicine is formulated to be administered by electrotransfer.

The invention relates to a method for obtaining an antiserum directed against a protein toxin by administering to an animal a solution comprising a genetic construct encoding a toxin immunogenic fragment, then applying an electric field in the administering zone, and isolating the serum. The invention also relates to the antiserum obtainable by the method as well as the use of the solution for the manufacture of a medicament for preventing or treating a toxic effect related to absorption by a mammal of a toxin, wherein said medicament is formulated to be administered by electrotransfer to the patient.

Today, the most common method for obtaining antisera against a protein antigen, for example a toxin or a poison, is to administer repeated injections of purified recombinant or native proteins in order to induce an immune response in the animal. Alternately, the protein can be expressed on a capsid or a viral envelope, or in a virosome. With regard to botulinum toxin and other lethal toxins, the entire toxin can not be used for immunization. Traditionally, the toxin must be produced from the bacterium and purified, then modified in order to inactivate its lethality while maintaining its antigenicity. This is achieved, for example, by purifying one of the toxin's incompletely functional subunits. Alternatively, one such recombinant subunit can be produced, for example using E. coli. This may prove essential in the absence of a reliable method for inactivating the toxin. In these two cases, producing inactivated native protein or recombinant fragments, the techniques are burdensome and costly. This explains why, for example, only one stock of multivalent serum against the various botulinum toxin serotypes have been produced to date.

An alternative pathway for obtaining an antiserum is genetic immunization, in which a DNA sequence encoding the toxin is administered to the animal to immunize. The coding DNA, in which the encoding gene is preceded by an adequate promoter and contains a polyadenylated sequence, can be carried either by a viral vector (adenovirus, AAV, retrovirus, lentivirus, etc.) or by a bacterial plasmid. It can also be produced by acellular synthesis in vitro, for example by PCR.

The ability to obtain immunization by injecting plasmid DNA was first demonstrated some ten years ago (Tang et al., Nature. 1992 Mar. 12; 356 (6365): 152-4; Ulmer et al., Science. 1993 Mar. 19; 259 (5102): 1745-9). Genetic immunization consists of injecting directly into skeletal muscle or skin, or into other tissues, the genes encoding antigenic proteins inserted into a circular fragment of bacterial DNA (plasmid). The organism itself produces antigens that can induce the immune reaction. It is now well established that immunization by DNA induces a long-lasting cellular and humoral response (Gurunathan et al., Annu Rev Immunol. 2000; 18:927-74. Review; Quinn et al., Vaccine. 2002 Aug. 19; 20 (25-26):3187-92).

Examples of recent publications reporting on this humoral response include:
A single intramuscular injection of plasmid encoding an HBV (hepatitis B virus) envelope protein causes antibody production for at least 74 weeks (Davis et al., Gene Ther. 1997 March; 4 (3):181-8), at a titer compatible with effective protection.
When a plasmid encoding a mutated Kunjin virus genome is injected via intramuscular route in mice, antibodies are produced with a titer that varies from 10 to 40. If these mice are exposed to wild Kunjin virus, or one highly similar to the West Nile virus, they are protected (0% to 20% mortality) (Hall et al., Proc Natl Acad Sci USA. 2003 Sep. 2; 100 (18):10460-4. Epub 2003 Aug. 13).
Intramuscular injection in mice of plasmids encoding the membrane region of human PSMA (prostate specific membrane antigen) protein led to the production of antibodies against this protein (Kuratsukuri et al., Eur Urol. 2002 July; 42 (1):67-73).

These examples show that it is possible to obtain neutralizing antibodies with satisfactory titers of the animal by DNA immunization. This is particularly true in mice, and the method is slightly less effective in larger animals (Babiuk et al., Vaccine. 2003 Jan. 30; 21 (7-8):649-58. Review; Dupuis et al., J Immunol. 2000 Sep. 1; 165 (5):2850-8).

A more highly effective transfer of genes can be achieved using physical techniques. For example, the ballistic “gene gun” method using DNA-covered gold particles projected into the animal's skin or mucous membrane at very high speed delivers DNA to the targeted cell nucleus. Another technique uses the ultrasound. Another technique, called the “hydrodynamic” or “hydrostatic” DNA injection method, uses the rapid intravenous or intra-arterial injection of a large volume of liquid containing encoding DNA, thus allowing the DNA to penetrate cells such as hepatocytes, endothelial cells or muscular cells, for example. Lastly, a highly effective physical method for administering DNA is electrotransfer, which the inventors developed at the laboratory. Electrotransfer is a simple and effective technique for transferring genes, consisting of injecting a DNA solution via intramuscular route followed by applying a series of electric pulses by means of electrodes connected to a generator (Aihara et al., Nat Biotechnol. 1998 September; 16 (9):867-70; Mir et al., C R Acad Sci III. November 1998; 321 (11):893-9; Mir et al., Proc Natl Acad Sci USA. 1999 Apr. 13; 96 (8):4262-7.). This method increases protein expression by several orders of magnitude (Lee et al., Mol Cells. 1997 Aug. 31; 7 (4):495-501; Kirman et al., Curr Opin Immunol. 2003 August; 15 (4):471-6. Review).

Several recent studies show the advantage of the electrotransfer technique in DNA immunization: for example, the titer of antibody produced increases by a factor of 100 in mice after electrotransfer of a plasmid encoding a HBV virus surface antigen (Widera et al., J Immunol. 2000 May 1; 164 (9):4635-40). This increase factor is roughly 10 in rabbits and guinea pigs. High antibody titers were also obtained in mice and rabbits after intramuscular electrotransfer of a plasmid encoding a hepatitis C virus envelope glycoprotein (Zucchelli et al., J Virol. 2000 December; 74 (24): 11598-607), and in mice after electrotransfer of a plasmid encoding a Mycobacterium tuberculosis protein (Tollefsen et al., Vaccine. 2002 Sep. 10; 20 (27-28):3370-8). This technique can also be applied to larger animals such as goats or bovines, (Tollefsen et al., Scand J Immunol. 2003 March; 57 (3):229-38). The inventors themselves have shown in the laboratory that electrotransfer of a plasmid encoding influenza hemagglutinin induced a better immune response in mice than intramuscular injection alone (Bachy et al., Vaccine. 2001 Feb. 8; 19 (13-14):1688-93). Lastly, it can be noted that it is possible to generate monoclonal antibodies against mite allergens in mice after immunization by electrotransfer (Yang et al., Clin Exp Allergy. 2003 May; 33 (5):663-8).

The electrotransfer technique is simple, easy to perform, and does not require the purification of recombinant proteins, generally a long, tedious and costly step required during conventional immunization. As a result, several epitopes can be tested quickly.

The genetic immunization techniques cited above (ballistic, ultrasonic, hydrodynamic, hydrostatic and electric methods) can be combined with conventional protein immunization methods. For example, an initial genetic immunization can be followed after several weeks with 1 to 2 genetic immunizations, followed finally after several weeks or months with several protein immunizations against the same antigen. Alternately, it is possible to first vaccinate against the protein and then perform genetic immunization.

The botulinum (Clostridium botulinum) and tetanus (Clostridium tetani) neurotoxins have a common organization. They are synthesized in the form of a single protein chain (˜150 kDa), which is then activated by proteolytic cleavage which produces two protein chains: the N-terminal light (L) chain (˜50 kDa) and the C-terminal heavy (H) chain (˜100 kDa), which remain joined by a disulfide bridge. Three functional domains have been defined on these neurotoxins. The C-terminal moiety of the H chain (called Hc) is the recognition domain for a receptor specific to the neuron surface. The N-terminal moiety of the H chain (H-N) is implicated in neuronal L chain uptake. The L chain contains the enzymatic proteolysis site for SNARE proteins and is responsible for neurotoxin intraneuronal activity, expressed as the blocking of neuroexocytosis. Each of these three functional domains is associated with a specific three-dimensional structure. The Hc domain contains two structures rich in beta sheets, the H-N domain is made of two very long alpha helices, and the L chain forms a compacts structure rich in beta sheets (Kozaki et al., Infect Immune. 1986 June; 52 (3):786-91; Kozaki et al., Infect Immune. 1987 December; 55 (12):3051-6).

All botulinum and tetanus neurotoxin genes have been sequenced and the crystallographic structure has been established for the botulinum A and B neurotoxins and the tetanus neurotoxin.

A variety of research was undertaken to determine the immunogenic fragment of these neurotoxins. It was shown initially that the Hc fragment of the tetanus toxin, obtained by papain proteolysis and purified by chromatography, is nontoxic and by anti-Hc immunization protects mice against a test dose of toxin (Kozaki et al., Infect Immune. 1989 September; 57 (9):2634-9.). This fragment subsequently was produced as a recombinant protein in Escherichia coli and was also shown to be an excellent immunogen (Halpern et al., Infect Immun. 1989 January; 57 (1):18-22.).

Among all the recombinant fragments of botulinum A neurotoxin tested, the only one that induces complete protection in mice is the heavy chain C-terminal domain, which corresponds to the tetanus neurotoxin Hc domain (Clayton et al., Infect Immune. 1995 July; 63 (7):2738-42; Dertzbaugh and West, Vaccine. November 1996; 14 (16):1538-44; Kubota et al., Appl Environ Microbiol. 1997 April; 63 (4): 1214-8; LaPenotiere et al., Toxicon. October 1995; 33 (10):1383-6. Review). All of the neutralizing monoclonal antibodies obtained with the whole botulinum A neurotoxin as immunogen were directed against the Hc fragment. Analysis of antibodies generated by vaccination with formalized whole botulinum neurotoxin in human showed that the majority were directed against the light chain and few against the Hc fragment. This study concluded that a vaccine based on the Hc fragment offers more protection than a vaccine prepared with the whole toxin (Brown et al., Hybridoma. October 1997; 16 (5):447-56). Thus, the second generation anti-botulinum vaccine developed by USAMRIID consists of recombinant, purified Hc fragments from seven botulinum neurotoxin types (A, B, C, D, E, F and G).

It was noted that the recombinant Hc fragment would be more effective than the corresponding anatoxin prepared conventionally. Protection using neutralizing neurotoxin antibodies primarily consists of blocking the cellular recognition receptor with the Hc fragment (Brown et al., 1997).

In addition, much research was performed to obtain neutralizing monoclonal antibodies against botulinum neurotoxins. Tests conducted with whole botulinum A neurotoxin often proved fruitless whereas those produced by immunizing mice with recombinant Hc protein yielded a significant number of neutralizing monoclonal antibodies (Amersdorfer et al., Infect Immun. 1997 September; 65 (9):3743-52; Middlebrook, Adv Exp Med Biol. 1995; 383:93-8). Thus, the Hc fragment proves to be a better immunogen than the whole, detoxified neurotoxin to induce neutralizing antibodies.

The method currently in use involves producing native or recombinant proteins, a long and expensive process. Moreover, if the native or recombinant protein is toxic, it must be denatured before injection into animals. The result may be antisera with weak neutralizing strength since only epitope antibodies can be obtained.

Thus, today there is a genuine need for protective antisera against botulinum toxins (or others), most notably in the event of bioterrorism.

The inventors have developed a novel method for obtaining an antiserum directed against a protein toxin, the antiserum obtained with this method having a higher titer in neutralizing antibodies against botulinum toxins. The novel method also has the advantages of being easy to implement and inexpensive.

Thus, according to a first aspect, the invention relates to a method for obtaining an antiserum directed against at least one protein toxin, comprising the following steps:

a) obtaining a solution comprising at least one genetic construct, said construct comprising a nucleic acid encoding at least one immunogenic fragment of said toxin,

b) administering by injection in an animal the solution obtained in step (a),

c) applying an electric field in the injection zone, and

d) subsequently withdrawing whole blood and isolating the serum.

The term “protein toxin” means any substance of animal, plant or bacterial origin that produces toxic effects and that is generally antigenic. “Protein toxin immunogenic fragment” means any fragment of said toxin with the capacity to induce an immune reaction or response.

The terms “protein,” “polypeptide” and “peptide” are used interchangeably in the present description to indicate a sequence of amino acids, or derivatives thereof, containing a sequence of amino acids.

In the sense of the present application, “subsequently sampling” (step (d)) means sampling with a minimum delay after step (c) (applying an electric field), which is required for immunization. Generally, this delay is at least 15 days after applying the electric field.

For the practical application of the present invention, a number of conventional molecular biology, microbiology and genetic engineering techniques are used. These techniques are well known and are explained, for example, in Current Protocols in Molecular Biology, Volumes I, II and III, 1997 (F. M. Ausubel, Ed.); Sambrook et al., 1989, Molecular Cloning: A Laboratory Manual, 2nd edition, Cold Spring Harbor Laboratory Press, Cold Spring Harbor, N.Y.; DNA Cloning: A Practical. Approach, Volumes I and II, 1985 (D. N. Glover, Ed.); Oligonucleotide Synthesis, 1984 (M. 1. Gait, Ed.); Nucleic Acid Hybridization, 1985 (Hames and Higgins); Transcription and Translation, 1984 (Hames and Higgins, Eds.); Animal Cell Culture, 1986 (R. I. Freshney, Ed.); Immobilized Cells and Enzymes, 1986 (IRL Press); Perbal, 1984, A Practical Guide to Molecular Cloning; the series, Methods in Enzymology (Academy Press, Inc.); Gene Transfer Vectors for Mammalian Cells, 1987 (J. H. Miller and M. P. Calos, Eds., Cold Spring Harbor Laboratory); and Methods in Enzymology Vol. 154 and Col. 155 (Wu and Grossmann, and Wu, Eds., respectively).

The conditions for applying an electric field in the injection zone according to step (c) are now well known to those persons skilled in the art, and are in particular described in the international patent applications published on Jan. 14, 1999, under the numbers WO 99/01157 and WO 99/01158. Those persons skilled in the art will be able to adapt these conditions according to each case.

Preferably, the electric field has an intensity between 1 and 800 V/cm in the form of 1 to 100,000 square impulses with a duration greater than 100 microseconds and with a frequency between 0.1 and 1,000 hertz. More preferably, the electric field has an intensity between 80 and 250 V/cm in the form of 1 to 20 square impulses with a duration between 1 and 50 milliseconds and a frequency of 1 to 10 hertz.

Advantageously, the injection is an intradermal or intramuscular injection.

According to a preferred embodiment, step (b) of administering the solution is preceded by a step of injecting a solution containing an enzyme that breaks down the extracellular matrix, such as hyalurdnidase. Indeed, this enzyme is responsible for breaking down hyaluronic acid, a major component of muscle extracellular matrix. Thus, hyaluronidase makes muscle cells more accessible to plasmids. Preferably, between 5 and 200 μl of a solution containing between 0.1 and 2 U/μl of hyaluronidase are injected. More preferably still, approximately 25 μl of a 0.4 U/μl solution of hyaluronidase in NaCl are injected.

Advantageously, the toxin is selected from the group comprised of Clostridium botulinum toxin, Clostridium tetani toxin, Bacillus anthracis toxin, ricin, diphtheria toxin and cholera toxin.

Still more advantageously, the immunogenic fragment of said toxin is the C-terminal fragment (Hc) selected from the group comprised of the Clostridium botulinum A serotype toxin Hc fragment of sequence SEQ ID NO 1, the Clostridium botulinum B serotype toxin Hc fragment of sequence SEQ ID NO 2, the Clostridium botulinum C serotype toxin Hc fragment of sequence SEQ ID NO 3, the Clostridium botulinum D serotype toxin Hc fragment of sequence SEQ ID NO 4, the Clostridium botulinum E serotype toxin Hc fragment of sequence SEQ ID NO 5, the Clostridium botulinum F serotype toxin Hc fragment of sequence SEQ ID NO 6, the Clostridium botulinum G serotype toxin Hc fragment of sequence SEQ ID NO 7, and the Clostridium tetani toxin Hc fragment of sequence SEQ ID NO 8, as well as variants thereof.

Preferably, the nucleic acid encoding the Clostridium botulinum A toxin Hc fragment is of sequence SEQ ID NO 17, or a variant thereof.

In its broadest sense, the term “variant” of a protein sequence indicates a sequence with modifications at the amino acid or nucleotide level only, with no influence on its functioning by decreasing its immunogenicity. In the same way, when used here in reference to a nucleotide sequence, “variant” means a nucleotide sequence corresponding to a reference nucleotide sequence, the corresponding sequence encoding a polypeptide having approximately the same structure and the same function as the polypeptide>encoded by the reference nucleotide sequence. It is desirable that the approximately similar nucleotide sequence encodes for the polypeptide encoded by the reference nucleotide sequence. It is desirable that the percent identity between the approximately similar nucleotide sequence and the reference nucleotide sequence is at least 90%, more preferably at least 95%, still more preferably at least 99%. Sequences are compared using the Smith-Waterman sequence alignment algorithm (see, for example, Waterman, M. S., Introduction to Computational Biology: Maps, sequences and genomes. Chapman & Hall. London: 1995. ISBN 0412-99391-0 or at http://www-hto.usc.edu/software/seqaln/index.html). The program localS version 1.16 is used with the following parameters: “match”: 1, “mismatch penalty”: 0.33, “open-gap penalty”: 2, “extended-gap penalty”: 2. A nucleotide sequence “approximately similar” to the reference nucleotide sequence hybridizes with the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., still more desirably in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C., and encodes for a functionally equivalent gene product.

According to another preferred embodiment, the genetic construct includes at the 5′ end of the nucleic acid encoding at least one fragment of said toxin, the cytomegalovirus (CMV) promoter.

The structure of the CMV promoter is described in particular in Hennighausen et al. (EMBO J. 5(6), 1367-1371, 1986).

According to another preferred embodiment, the genetic construct includes a sequence encoding an extracellular secretion signal. These extracellular secretion signals, which are well known to those persons skilled in the art, make it possible to obtain higher antibody titers.

Preferably, the sequence encoding the extracellular secretion signal is selected among sequences SEQ ID NO 9, which encodes for the mouse erythropoietin extracellular secretion signal, and SEQ. ID NO 10, which encodes for the human alkaline phosphatase extracellular secretion signal, or a variant thereof.

According to still another preferred embodiment, the genetic construct includes at the 5′ end of the promoter a translation initiation site nucleic sequence, a so-called KOZAK sequence, of sequence SEQ ID NO 11.

According to still another preferred embodiment, at least one initial codon of the nucleic acid sequence that encodes for at least one fragment of said toxin is replaced by a different codon encoding the same amino acid and whose frequency in eukaryotic cells is greater than their frequency in Clostridium botulinum, as defined in Table 1.

TABLE 1
Codon frequency (%)

| | | | | | | | | | | | | |
|---|---|---|---|---|---|---|---|---|---|---|---|---|
| Genome: | UUU | 15.5 | 45.4 | UCU | 10.7 | 23.6 | UAU | 12.6 | 54.5 | UGU | 10.0 | 6.8 |
| Mus musculus | UUC | 23.8 | 6.1 | UCC | 14.2 | 3.1 | UAC | 17.8 | 5.9 | UGC | 12.0 | 1.7 |
| Clostridium botulinum | UUA | 6.5 | 55.5 | UCA | 15.1 | 22.0 | UAA | 0.7 | 1.1 | UGA | 1.1 | 0.0 |
| | UUG | 9.0 | 8.4 | ACG | 4.2 | 1.4 | UAG | 1.1 | 0.3 | UGG | 15.8 | 11.5 |
| | CUU | 11.8 | 11.4 | CCU | 14.3 | 13.7 | CAU | 11.4 | 5.7 | CGU | 3.3 | 2.6 |
| | CUC | 18.4 | 0.9 | CCC | 16.5 | 2.0 | CAC | 22.2 | 0.8 | CGC | 7.0 | 0.3 |
| | CUA | 13.0 | 8.7 | CCA | 18.8 | 13.2 | CAA | 16.5 | 26.9 | CGA | 5.3 | 1.1 |
| | CUG | 30.3 | 0.7 | CCG | 5.8 | 1.0 | CAG | 49.8 | 4.2 | CGG | 6.9 | 0.2 |
| | AUU | 15.9 | 44.4 | ACU | 13.0 | 23.8 | AAU | 18.7 | 103.7 | AGU | 8.9 | 24.2 |
| | AUC | 25.4 | 5.3 | ACC | 15.2 | 2.9 | AAC | 26.4 | 12.0 | AGC | 15.9 | 5.1 |
| | AUA | 18.1 | 54.7 | ACA | 23.3 | 22.8 | AAA | 46.8 | 62.6 | AGA | 20.1 | 20.3 |
| | AUG | 22.7 | 16.2 | ACG | 4.4 | 2.1 | AAG | 30.1 | 14.7 | AGG | 14.0 | 3.3 |
| | GUU | 5.8 | 21.0 | GCU | 12.4 | 15.0 | GAU | 18.2 | 53.7 | GGU | 9.1 | 13.7 |
| | GUC | 10.0 | 1.1 | GCC | 19.4 | 1.9 | GAC | 28.9 | 6.0 | GGC | 17.5 | 2.7 |
| | GUA | 8.3 | 21.9 | GCA | 17.8 | 15.2 | GAA | 31.7 | 49.3 | GGA | 15.7 | 21.0 |
| | GUG | 17.6 | 3.1 | GCG | 6.2 | 1.2 | GAG | 30.4 | 10.0 | GGG | 10.8 | 4.2 |

Since botulinum toxins are produced naturally by Clostridium, the genetic code used by this organism is not necessarily adapted to satisfactory expression of the protein in mammals. Thus, the inventors have used a synthetic gene designed according to the codon optimization technique, i.e., using synonymous codons corresponding to the most common eukaryotic cell tRNA (transfer RNA).

According to another preferred embodiment, the genetic construct also includes a nucleic acid encoding at least one cytokine.

Advantageously, the solution in step (a) includes another genetic construct that contains a nucleic acid encoding a cytokine, said two genetic constructs being co-administered in step (b).

Preferably, the sequence of the nucleic acid encoding the cytokine is selected from the group comprised of SEQ ID NO 12, which encodes for the hematopoietic growth promoter (GM-CSF), SEQ ID NO 13, which encodes for mouse interleukin 12 subunit p35, SEQ ID NO 14, which encodes for mouse interleukin 12 subunit p40, SEQ ID NO 15, which encodes for mouse interleukin 4, and SEQ ID NO 16, which encodes for human interleukin 10.

According to another advantageous embodiment, the genetic construct also includes a non-methylated immunostimulation sequence rich in guanine and cytosine bases, between 10 and 10,000 nucleotides in size.

One such sequence, the so-called CpG sequence, is well known to those persons skilled in the art. It must be understood that in the present invention the immunostimulation sequence can also be a specific oligonucleotide which will be co-administered with the plasmid encoding the toxin fragment. (Mutwiri et al., Veterinary Immunology and Immunopathology, 2003, 91, 89-103; R. Rankin, et al., Vaccine 2002, 20, 3014-3022).

According to a particularly advantageous embodiment, the antiserum is directed against at least two protein toxins and the solution in step (a) comprises a mixture of at least two genetic constructs, each of said constructs comprising a nucleic acid encoding at least one immunogenic fragment of said toxins.

Preferably, the animal is selected among mice, rabbits, horses and pigs.

According to one particularly preferred embodiment, steps (b) and (c) are repeated at least once before step (d). Generally, these steps are repeated at an interval of at least 15 days, preferably at least three weeks, and in a particularly preferred way, at least one month.

Still more preferably, step (c) is followed by administering to the animal the recombinant immunogenic fragment of said toxin. Generally, this administration is conducted at least 15 days after step (c). The serum is then isolated in step (d).

The serum can be isolated by any method known to those skilled in the art. Preferably, the serum is isolated in step (d) by centrifugation.

According to a second aspect, the present invention relates to an antiserum directed against a protein toxin obtainable by the method described above, wherein its antitoxin antibody titer is equal to or greater than 100, and wherein its neutralizing strength is equal to or greater than 100.

The antibody titer can be determined by carrying out dilutions, for example doubling dilutions of sera starting from a 1/100 dilution, followed by an ELISA, which yields a plot of optical density at a given wavelength, for example at 492 nm when the peroxidase/ortho-phenylenediamine system is used, as a function of dilution. The antibody titer corresponds to the reciprocal of the dilution factor that gives an optical density of at least 0.2 above reference sera.

To determine neutralizing strength or neutralizing titer, the presence of neutralizing antibodies is determined by a test of lethality in mice: for example, botulinum A neurotoxin is produced and calibrated at 10 mouse lethal doses per ml. Serum dilutions are then incubated with a toxin preparation, and injected into mice. Mouse survival is then observed for a few days. The results are expressed in neutralizing units per ml (a neutralizing unit corresponds to the volume of serum that neutralizes 10 mouse lethal doses).

The invention also relates to the antiserum of the present invention, for the use thereof as a preventive serum or antidote to neutralize in a mammal the toxic effects related to the absorption of the toxin in said mammal.

In the present application, the absorption of toxin can result from the bacterial contamination of said mammal.

The present invention also relates to the use of the invention for the manufacture of a medicament for preventing or treating a toxic effect related to absorption by a mammal of a toxin selected from the group comprising Clostridium botulinum toxin, Clostridium tetani toxin, Bacillus anthracis toxin, ricin, diphtheria toxin and cholera toxin.

The invention also relates to the use of the antiserum of the present invention, as a reagent in an immunological test, such as, for example, without being in any way restrictive, immuno-enzymatic ELISA titration, immunotransfer, immunoluminescent titration, etc. Persons skilled in the art know these various immunological tests well and will be able to apply the antiserum of the invention to such tests.

According to a final aspect, the invention relates to the use of a solution containing at least one genetic construct according to the present invention, the manufacture of a medicament for preventing or treating a toxic effect related to absorption by a mammal of a toxin selected from the group comprised of Clostridium botulinum toxin, Clostridium tetani toxin, Bacillus anthracis toxin, ricin, diphtheria toxin and cholera toxin, wherein said medicament is formulated to be administered by electrotransfer.

Applicable electroporation conditions for administration by electrotransfer, as well as injection mode and number, are as previously defined.

The medicine, prepared from the solution containing said at least one genetic construct, must be formulated in the absence of cationic lipids to allow electrotransfer. It can be formulated in the presence of any pharmaceutically acceptable excipient known to those persons skilled, in the art, such as saline solution, phosphate buffer, glucose buffer, etc.

Preferably, the use according to the invention is characterized in that the solution also contains an immunostimulator adjuvant. Examples of immunostimulator adjuvants include, without being in any way restrictive, Freund's adjuvant and alum.

The following examples and figures serve to illustrate the present invention without, however, limiting its scope.

### FIGURE LEGENDS

The “*” symbol in certain figures corresponds to a antibody titer lower than 100.

FIG. 1: ELISA assay of sera three weeks after electrotransfer. Doubling dilutions of sera starting from a 1/100 dilution.

FIG. 2: ELISA assay of sera 70 days after electrotransfer. Doubling dilutions of sera starting from a 1/100 dilution.

FIG. 3: Antibody titers obtained from ELISA assays from 21 to 70 days after electrotransfer (antibody titer=reciprocal of the dilution factor that gives an OD₄₉₀ of 0.3 above reference sera).

FIG. 4: Comparison of injection alone/injection+electrotransfer with plasmids pVaxFcBoNTA and pVaxFc*BoNTA.

FIG. 5: Supply of the codon optimization at the FcBoNTA sequence (FcBoNTA/FC*BoNTA).

FIG. 6: Effect of hyaluronidase on antibody titer (plasmids pVaxFcBoNTA and pVaxFc*BoNTA).

FIG. 7: Effect of hyaluronidase on antibody titer (plasmids pVaxFc*BoNTA-Master).

FIG. 8: titers of anti-FcBoNTB antibody with plasmids pVaxFc*BoNTA and pVaxFc*BoNTA-Master (injection+electrotransfer).

FIG. 9: titers of anti-FcBoNTE antibody with plasmids pVaxFcBoNTE, pVaxFc*BoNTE, pVaxFc*BoNTE-Master and pVaxFc*BoNTE-Variant.

FIG. 10: titers of anti-FcBoNTA, anti-FcBoNTB and anti-FcBoNTE antibodies in ABE (with plasmids pVaxFc*BoNTA-Master, pVaxFc*BoNTB-Master and pVaxFc*BoNTE-Master co-injected and electrotransferred: ABE multivalent serum).

FIG. 11: titers of anti-FcBoNTA antibody in rabbits.

FIG. 12: titers of anti-FcBoNTA antibody with or without re-injection of plasmid pVaxFc*BoNTA-Master in mice (“id.” for intradermal and “im.” for intramuscular).

FIG. 13: titers of anti-FcBoNTA antibody with or without re-injection of the plasmid pVaxFc*BoNTA in mice.

FIG. 14: Advantage of codon optimization to obtain higher antiserum titers: using pVaxFcNoNTA (dark) or the optimized codon sequence of the botulinum A serotype toxin Hc fragment (plasmid A pVaxFc*BoNTA, grid pattern).

FIG. 15: Obtaining antisera by the method of the invention, assayed three times after electrotransfer, using an optimized genetic sequence encoding a fragment of botulinum A toxin, unassociated (FC*BoNTA) or associated (secreted FC*BoNTA, grid pattern column) with a protein secretion sequence.

FIG. 16: Obtaining antisera by the method of the invention, assayed three times after electrotransfer, using an optimized genetic sequence encoding a fragment of botulinum B toxin, unassociated (FC*BoNTB) or associated (secreted FC*BoNTB) with a protein secretion sequence.

FIG. 17: Obtaining antisera by the method of the invention, assayed three times after electrotransfer, using an optimized genetic sequence encoding a fragment of botulinum E toxin:
not codon-optimized (FcBoNTE),
codon-optimized, unassociated or associated with a protein secretion sequence (FC*BoNTE),
codon-optimized and associated or associated with a protein secretion sequence (secreted FC*BoNTE)

### EXAMPLES

### I—Materials and Methods

Genetic Material

The inventors injected and electrotransferred various plasmid constructs encoding the C-terminal fragment of botulinum A toxin, hereafter referred to as FcBoNTA, a fragment known to be the most immunogenic region of the toxin. The various constructs tested are:
pVaxFcBoNTA: this plasmid contains the FcBoNTA fragment under control of a CMV promoter.
pVaxFc*BoNTA: this plasmid contains the FcBoNTA fragment whose sequence was optimized for optimal protein expression in mice (designated FC*BoNTA). Indeed, codon frequencies in Clostridium botulinum and in mice are very different: this indicates that the pool of transfer RNA in these two species is different, which may be a limiting factor. The sequence was entirely modified to yield the same protein using the most common codons in mice. The Fc* fragment is under the control of a CMV promoter.
pVaxFc*BoNTA-Master: this plasmid contains the FC*BoNTA fragment fused with the murine erythropoietin secretion signal, and is preceded by a Kozak sequence which improves translation.
pVaxFc*BoNTA-Variant: this plasmid contains the FC*BoNTA fragment fused with the human secreted alkaline phosphatase secretion signal, and is preceded by a Kozak sequence which improves translation.

Procedure

These various constructs were injected and electrotransferred in SWISS mice at a dose of 40 μg per injection:
in 30 μl of 150 mM NaCl in the cranial tibial muscle,
in 100 μl of 150 mM NaCl in the skin via intradermal route.

In all cases, the procedure is as follows: the mice are anesthetized (intraperitoneal injection of a Ketamine/Xylazine mixture), their hind paws are shaved, and then the plasmid solution is injected into the cranial tibial muscle or the skin. The muscle or skin is then exposed to a 200 V/cm electric field in the form of eight 20 ms square impulses of 2 Hz in frequency using two electrode plates connected to a Genetronics EC 830 electric generator. If necessary, a hyaluronidase solution (25 μl at 0.4 U/μl in 150 mM NaCl) is injected into the cranial tibial muscle two hours before injection and electrotransfer.

Blood (approximately 150 μl) from anesthetized mice is sampled via a retro-orbital puncture. For serum assays the samples are centrifuged at 3,000 rpm for 10 minutes at 4° C. Plasma is collected and the sera are preserved at −80° C.

Anti-FcBoNTA, Anti-FcBoNTB and Anti-FcBoNTE Antibody Assays (ELISAs)

An ELISA is performed to assay anti-FcBoNTA (or anti-FcBoNTB or anti-FcBoNTE) antibodies in mice sera. In practical terms, the recombinant FcBoNTA, FcBoNTB or FcBoNTE protein is deposited at the bottom of well in a 96-well plate and the sera are then incubated with the plate: if antibodies are present in the serum, they will bind to the protein. Washes remove everything not bound to the recombinant protein and the presence of anti-Fc antibody is then detected by the combination of a secondary biotinylated mouse anti-Ig antibody and streptavidin coupled with peroxidase. The plate is then developed with a peroxidase substrate and read at 492 nm.

To determine antibody titer, doubling dilutions of the sera are prepared starting with a 1/100 dilution. The plot of optical density at 492 nm as a function of dilution is used to determine the antibody titer corresponding to the reciprocal of the dilution factor that gives an OD₄₉₀ of 0.3 above reference sera.

Neutralizing Antibody Assay (Lethality Test)

The presence of neutralizing antibodies is determined by a mice lethality test: botulinum A neurotoxin is produced and calibrated at 10 mouse lethal doses per ml. Serum dilutions are then incubated with 2 ml of toxin preparation for 30 minutes at 37° C., and injected into mice by intraperitoneal route (two mice per dilution, 1 ml per mouse). Mouse survival is then observed for four days. The results are expressed as neutralizing units per ml (one neutralizing unit corresponds to the volume of serum that neutralizes 10 mouse lethal doses).

### II—Additional Experiments

1) Comparison of Injection Alone/Injection+Electrotransfer

The inventors conducted an experiment to validate the advantage of electrotransfer. To that end, the inventors compared the antibody titers obtained from batches of mice injected with the same plasmid (pVaxFcBoNTA or pVaxFc*BoNTA) but with or without electrotransfer following injection.

The antibody titers obtained 30 days after treatment are given in FIG. 4.

Following this experiment, the inventors tested the neutralizing strength of these antibodies obtained by injection alone or by injection+electrotransfer: the inventors thus conducted a neutralization test, i.e., a lethality test, in mice. The sera were tested at 45 days and sera from identical treatments were pooled to limit the number of mice used.

The results presented in table 2 give the number of living mice of the number of total mice for each serum dilution and each treatment. The neutralizing titer is deduced therefrom as the reciprocal of the highest dilution at which the mice remain alive:

TABLE 2

| | Dilutions | | | | Neutralizing titer |
|---|---|---|---|---|---|
| Fc*BoNTA | 10⁻² | 10⁻³ | 10⁻⁴ | 10⁻⁵ | *10 MLD |
| Injection alone | 0/2 | 0/2 | 0/2 | 0/2 | <100 |
| Injection + | 2/2 | 1/2 | 0/2 | 0/2 | 1,000 |
| electrotransfer | | | | | |

Thus it is noted that the antibodies obtained with an injection alone are not neutralizing whereas with electrotransfer the results are comparable with those observed previously.

1) Various Comparisons

a) Effect on Optimization:

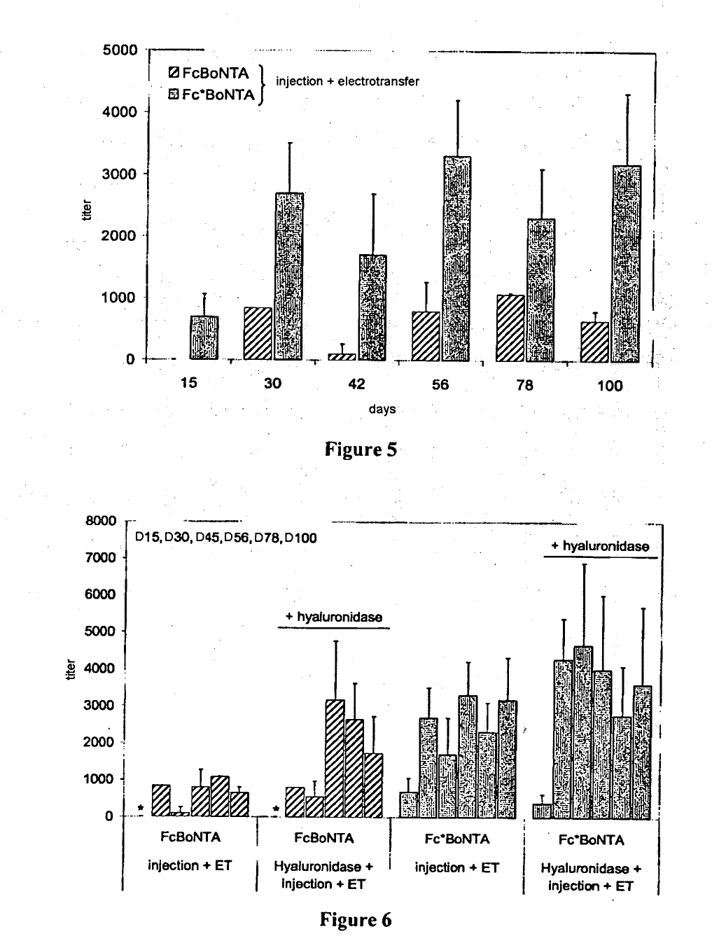

The inventors compared the supply of codon optimization at the sequence administered by electrotransfer (FIG. 5) or without electrotransfer (FIG. 4).

It is clearly observed that codon optimization in the FcBoNTA sequence very strongly increases antibody titer (grayed compared to hatched).

b) Effect of Hyaluronidase in the Electrotransfer Method

The inventors studied the effect of hyaluronidase on antibody titer:

The results obtained with the pVaxFcBoNTA plasmid are presented in FIG. 6.

The results obtained with the pVaxFc*BoNTA plasmid are presented in FIG. 6.

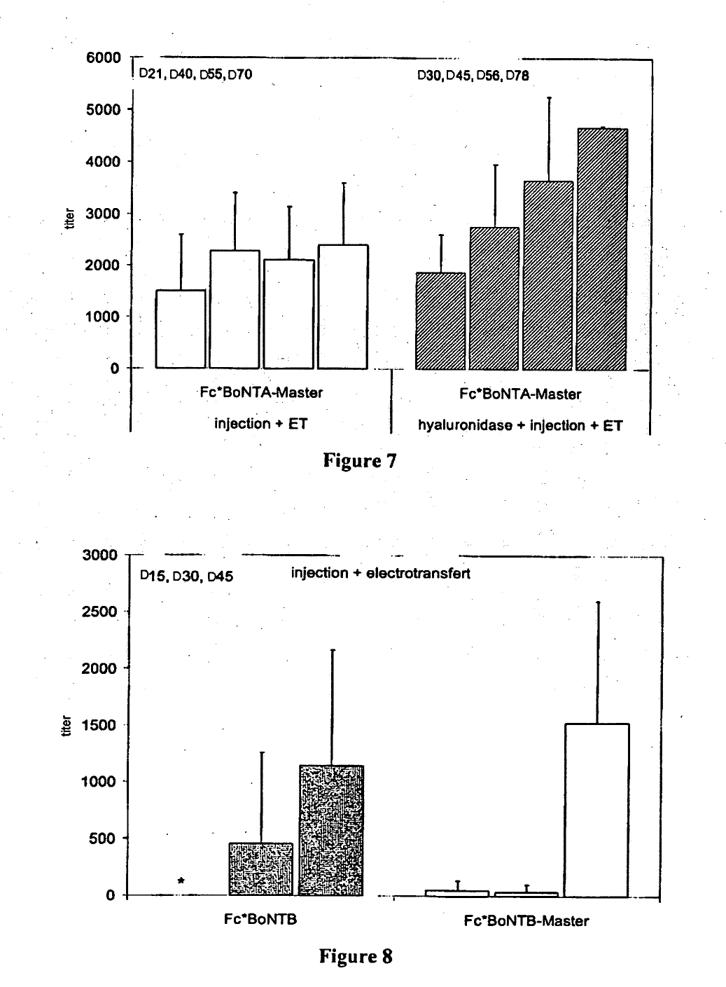

The results obtained with the pVaxFc*BoNTA-Master plasmid are presented in FIG. 7.

2) Toxins B and E:

The inventors followed the exact protocol as with toxin A.

Injection+electrotransfer of 40 μg of pVaxFc*BoNTB plasmid and pVaxFc*BoNTB-Master (C-terminal BoNTB fragment+Epo secretion signal+Kozak sequence).

Samples were taken at 15, 30 and 45 days after injection and electrotransfer.

The results obtained for the anti-FcBoNTB antibody titers are presented in FIG. 8.

Thus it is possible to obtain anti-FcBoNTB antibodies by plasmid electrotransfer.

Anti-FcBoNTE Antibody Titer

Same protocol as with toxin E (40 μg of plasmid).

The inventors compared:
pVaxFcBoNTE: non-secreted, non-optimized C-terminal fragment,
pVaxFc*BoNTE: optimized C-terminal fragment (codons)
pVaxFc*BoNTE-Master: optimized C-terminal fragment+Epo secretion signal+Kozak sequence
pVaxFc*BoNTE-Variant: optimized C-terminal fragment+hSeAP secretion signal+Kozak sequence

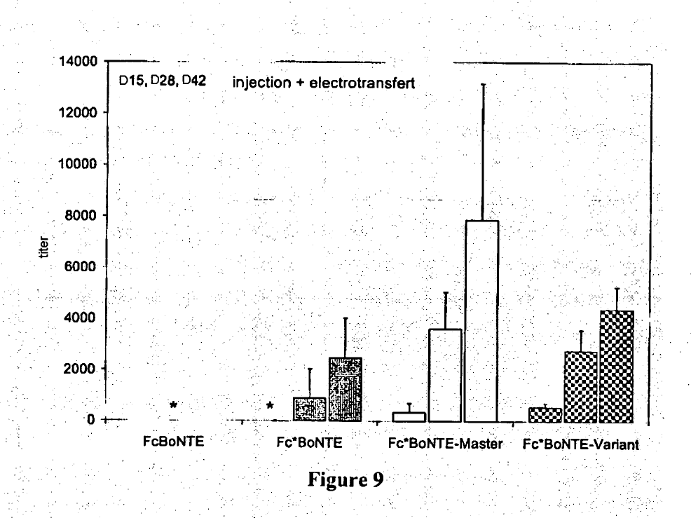

Samples were taken at 15, 28 and 42 days. The results are presented in FIG. 9.

3) Multivalent Sera:

The inventors tested the co-injection+electrotransfer of several plasmids encoding several C-terminal fragments: FcBoNTA, FcBoNTB, and FcBoNTE.

The three plasmids encode for the C-terminal fragments preceded by the mouse Epo secretion signal and a Kozak sequence.

40 μg of each plasmid were injected (20 μg of each in each mouse paw) for a total of 60 μg of DNA per paw.

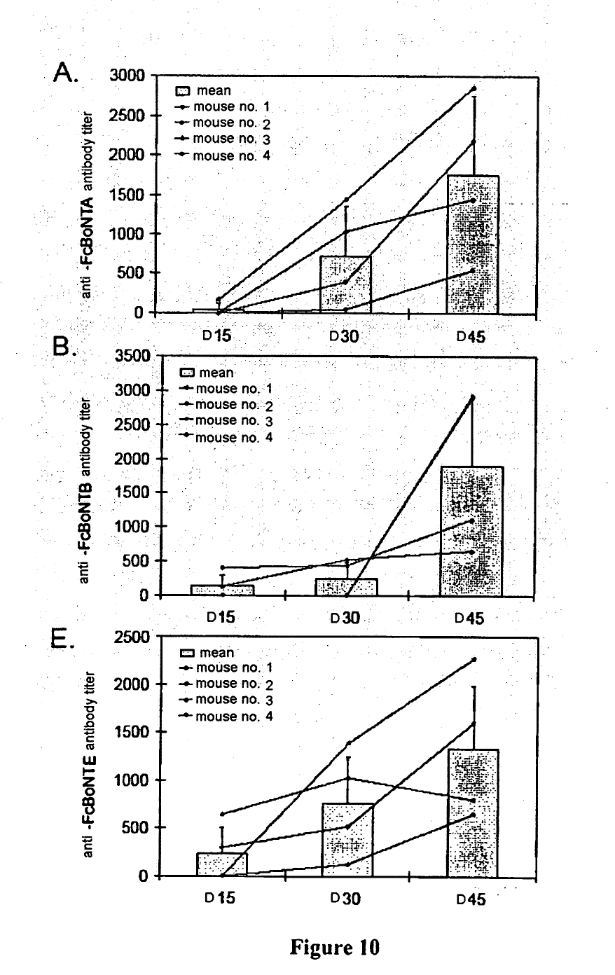

Anti-FcBoNTA antibody titers are presented in FIG. 10A.

Anti-FcBoNTB antibody titers are presented in FIG. 10B.

Anti-FcBoNTE antibody titers are presented in FIG. 10E.

4) In Rabbits:

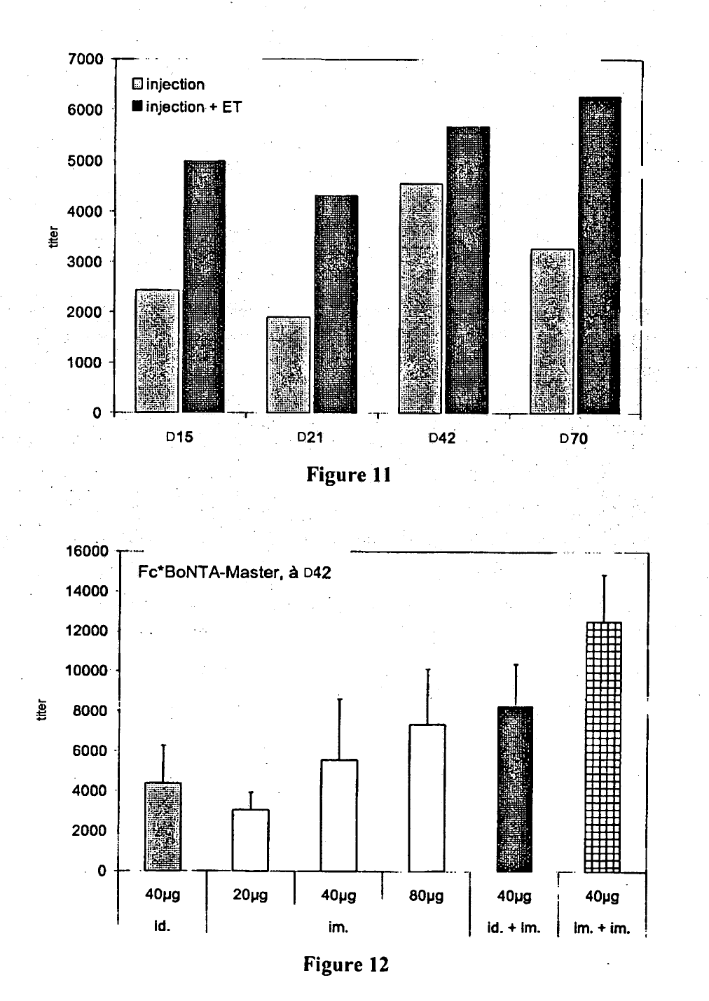

The inventors tested injection or injection+electrotransfer of 500 μg of pVaxFc*BoNTA-Master plasmid in rabbits. Electrotransfer conditions are as follows: eight 125 V/cm impulses of 20 ms at a frequency, of 2 Hz with needle electrodes. The results are presented in FIG. 11.

5) Effect of Re-Injections:

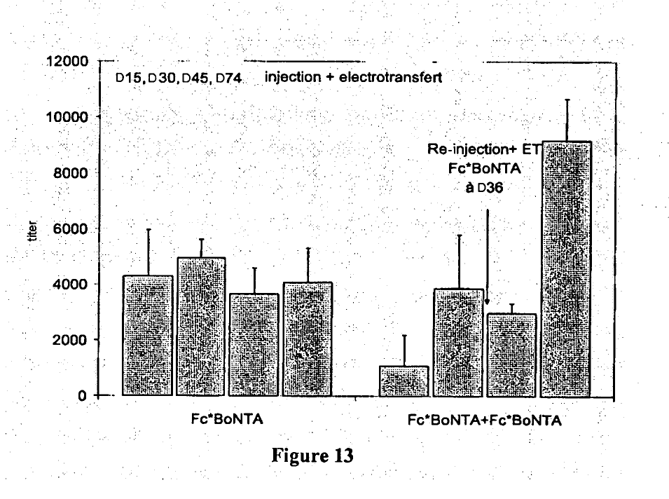

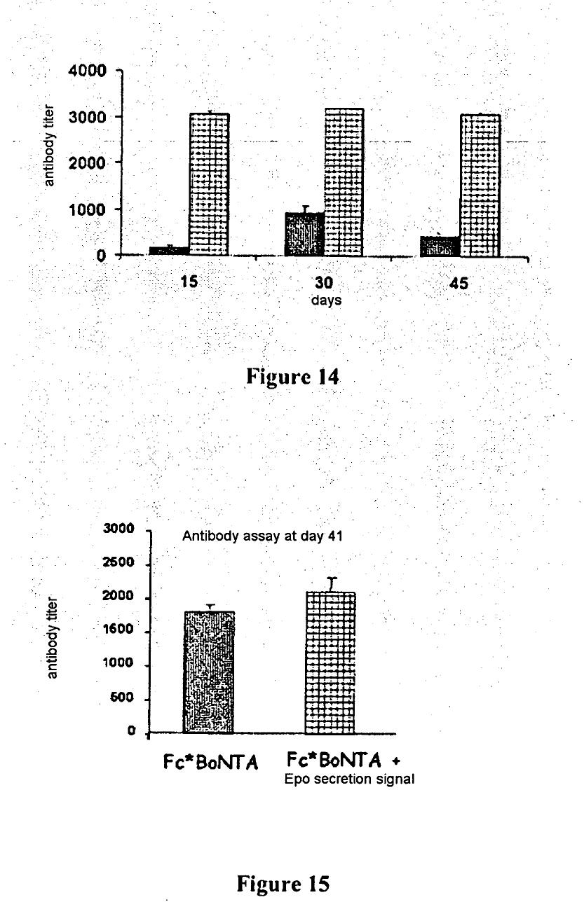

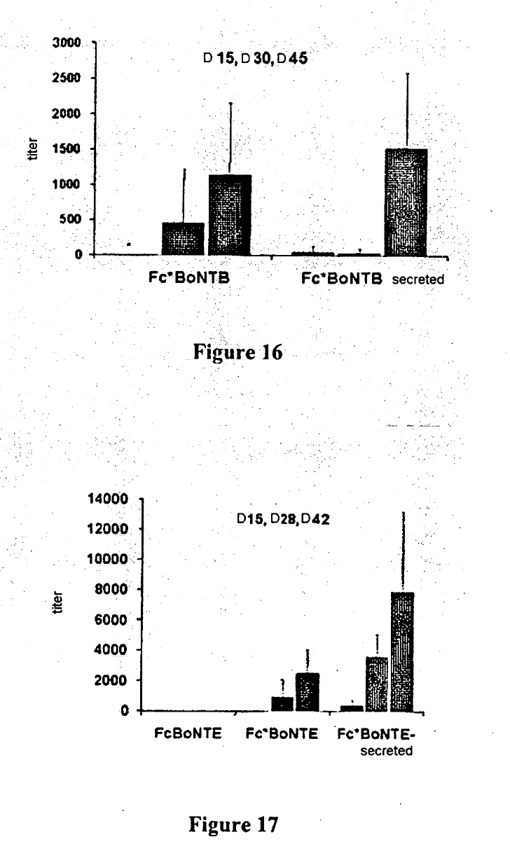

The inventors tested the effect of a second re-injection+electrotransfer in mice:
two injections+electrotransfer in each muscle at day 0 with the pVaxFc*BoNTA-Master plasmid (notation im. 80 μg) (FIG. 12)
two injections+electrotransfer with a three week interval via intramuscular route each time with the pVaxFc*BoNTA-Master plasmid (notation im.+im. 40 μg) (FIG. 12)
two injections+electrotransfer with a three week interval, the first treatment intradermal, the second intramuscular, with the pVaxFc*BoNTA-Master plasmid (notation id.+im. 40 μg) (FIG. 12)
two injections+electrotransfer with a one month interval via intramuscular route each time with the pVaxFc*BoNTA plasmid (FIG. 13)

### III—Results

The inventors compared various constructs and various procedures (four mice per treatment):
injection only (injection+electrotransfer)
intramuscular injection+electrotransfer of 40 μg of pVaxFcBoNTA
intramuscular injection+electrotransfer of 40 μg of pVaxFc*BoNTA (optimized sequence)
intramuscular injection+electrotransfer of 40 μg of pVaxFc*BoNTA-Master (optimized sequence+murine erythropoietin secretion signal+Kozak sequence)
intramuscular injection+electrotransfer of 40 μg of pVaxFc*BoNTA-Variant (optimized sequence+human alkaline phosphatase secretion signal+Kozak sequence)
intradermal injection+electrotransfer of 40 μg of pVaxFc*BoNTA (optimized sequence)
hyaluronidase treatment+injection+intramuscular electrotransfer of 40 μg of pVaxFc*BoNTA (optimized sequence)
no treatment

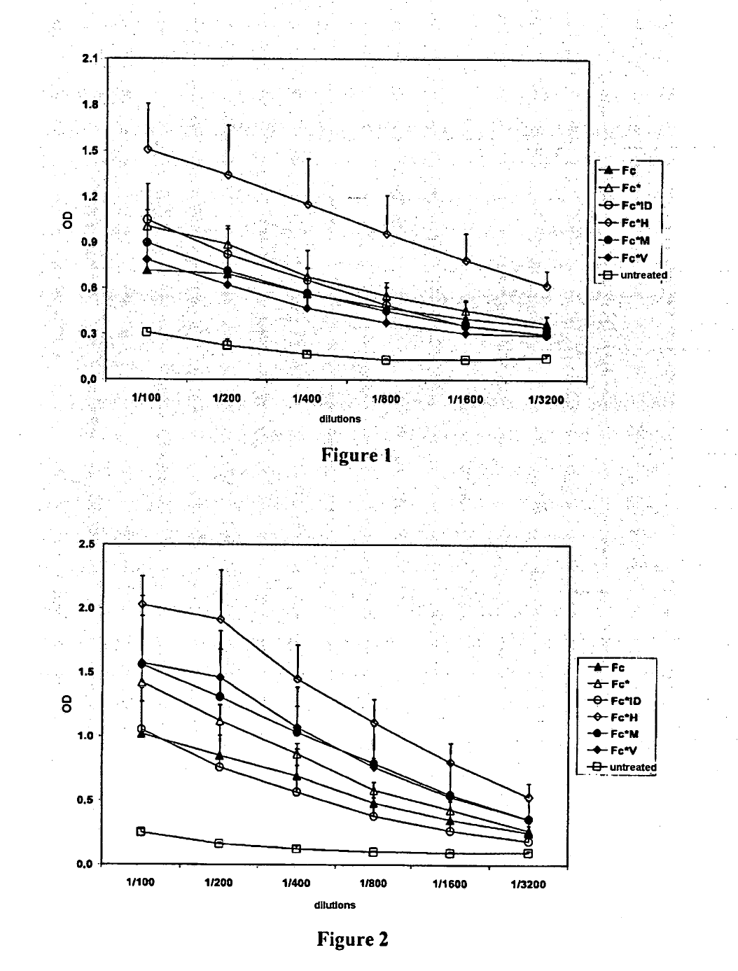

The results obtained with an ELISA assay of sera three weeks after electrotransfer are presented in FIG. 1.

Thus, after three weeks the inventors detect anti-FcBoNTA antibodies in all of the mouse sera treated under the various conditions described, and not in the sera of untreated mice. It can be noted, however, that antibody titer varies according to treatment: the mice treated with hyaluronidase have an antibody titer higher than the others. This enzyme is responsible for breaking down hyaluronic acid, a major component of the muscle extracellular matrix. Thus, hyaluronidase makes muscle cells more accessible to plasmids. Intradermal electrotransfer can also produce antibodies.

Samples were taken every 15 days; the ELISA assay results at 70 days after injection are presented in FIG. 2.

The ELISA assay results at 70 days resemble those obtained at 21 days. It can be noted, however, that antibody titers increased under all the treatment conditions except for the intradermal condition. This can be explained by the fact that the inventors showed that protein expression following intradermal electrotransfer lasts only about 15 days, as compared with muscle expression kinetics which persist for up to one year.

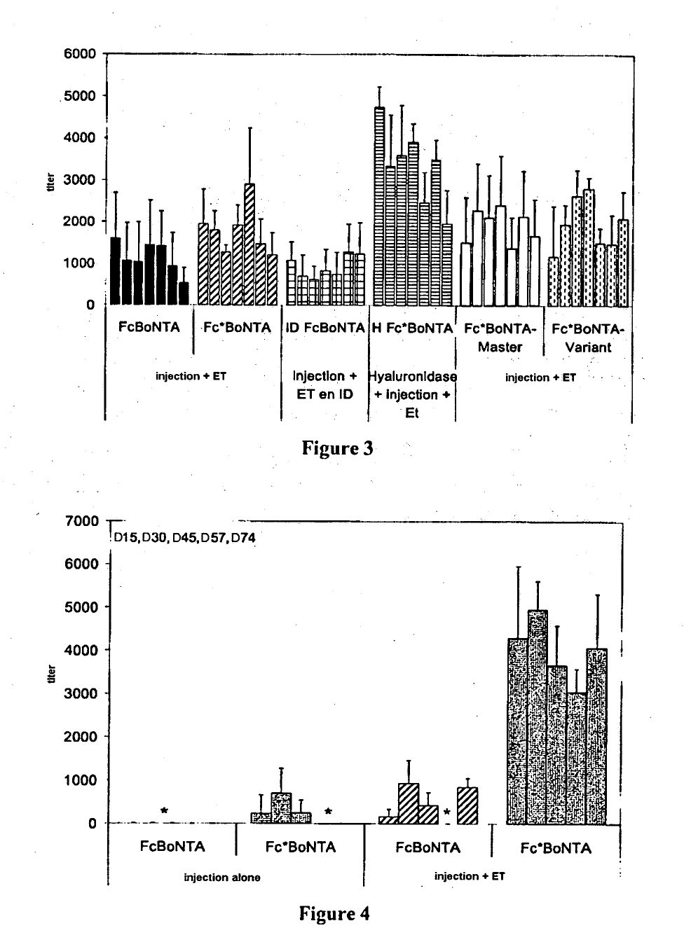

For an overall view of antibody titer kinetics, FIG. 3 presents all of the titers obtained per condition over time.

These results provide information about mouse serum antibody titer for each condition but do not provide information as to these antibodies' neutralizing strength. The inventors thus conducted a neutralization (lethality) test in mice. The sera taken at day 40 were tested and sera from the same condition were pooled to limit the number of mice used.

The results presented in Table 3 give the number of living mice of the number of mice treated with each serum dilution and each condition. The neutralizing titer is deduced as the inverse of the strongest dilution at which the mice remain alive:

TABLE 3
mice surviving after a lethal challenge
(10 lethal doses) of BoNTA toxin

| | | Dilutions | | | | Neutralizing titer |
|---|---|---|---|---|---|---|
| | | 10⁻² | 10⁻³ | 10⁻⁴ | 10⁻⁵ | *10 MLD |
| pVaxFc | Fc | 2/2 | 0/2 | 0/2 | 0/2 | 100 |
| pVaxFc* | Fc* | 2/2 | 0/2 | 0/2 | 0/2 | 100 |
| pVaxFc*Master | M | 2/2 | 2/2 | 2/2 | 0/2 | 10,000 |
| pVaxFc*Variant | V | 2/2 | 1/2 | 0/2 | 0/2 | 100-1,000 |
| pVaxFc* + hyalu | H | 2/2 | 2/2 | 0/2 | 0/2 | 1,000 |
| pVaxFc* | ID | 0/2 | 0/2 | 0/2 | 0/2 | 0 |
| intradermic | | | | | | |

The first conclusion of this test is that the antibodies obtained by plasmid electrotransfer are neutralizing.

The second conclusion is that certain conditions give a highly convincing neutralizing titer, with the pVAxFc*BoNTA-Master condition in particular giving a neutralizing titer of at least 10,000.

The inventors then conducted an experiment to validate the advantage of electrotransfer. To this end, they compared antibody titers obtained from batches of mice injected with the same plasmid (pVaxFcBoNTA or pVaxFc*BoNTA) but with or without electrotransfer following injection.

The antibody titers obtained 30 days after treatment are presented in FIG. 4.

In both cases a strong increase in antibody titer can be observed in the injection-AND electrotransfer batches compared with the injection-only batches.

### IV—Conclusion

The inventors obtained high neutralizing antibody titers after a simple injection and electrotransfer of plasmid encoding the botulinum A toxin C-terminal FcBoNTA fragment. This result suggests that it is possible by this simple method to obtain therapeutic monovalent or multivalent botulinum antitoxin antisera. Indeed, a multivalent antiserum can be obtained by genetic immunization with several plasmids because it has been shown that with the electrotransfer technique co-transfection leads to co-expression. Alternatively, a multivalent antiserum can be obtained by simply mixing univalent antisera.

1. A method for obtaining an antiserum directed against at least one protein toxin, comprising the following steps:
a) obtaining a solution comprising at least one genetic construct, said construct comprising a nucleic acid encoding at least one immunogenic fragment of said toxin,
b) administering by injection in an animal the solution obtained in step (a),
c) applying an electric field in the injection zone, and
d) subsequently sampling whole blood and isolating the serum.

2. A method according to claim 1, wherein the electric field has an intensity between 1 and 800 V/cm in the form of 1 to 100,000 square impulses with a duration greater than 100 microseconds and with a frequency between 0.1 and 1,000 hertz.

3. A method according to claim 2, wherein the electric field has an intensity between 80 and 250 V/cm in the form of 1 to 20 square impulses with a duration between 1 and 50 milliseconds and with a frequency between 1 and 10 hertz.

4. A method according to claim 1, wherein the injection is an intradermal or intramuscular injection.

5. A method according to claim 4, wherein step b of administering the solution is preceded by a step of injecting a solution containing an enzyme that breaks down the extracellular matrix.

6. A method according to claim 5, wherein between 5 and 200 μl of a solution containing an enzyme between 0.1 and 2 U/μl of hyaluronidase are injected.

7. A method according to claim 1, wherein the toxin is selected from the group consisting of Clostridium botulinum toxin, Clostridium tetani toxin, Bacillus anthracis toxin, ricin, diphtheria toxin and cholera toxin.

8. A method according to claim 7, wherein the immunogenic fragment of said toxin is the C-terminal fragment (Hc) selected from the group consisting of the Clostridium botulinum A serotype toxin Hc fragment of sequence SEQ ID NO 1, the Clostridium botulinum B serotype toxin Hc fragment of sequence SEQ ID NO 2, the Clostridium botulinum C serotype toxin Hc fragment of sequence SEQ ID NO 3, the Clostridium botulinum D serotype toxin Hc fragment of sequence SEQ ID NO 4, the Clostridium botulinum E serotype toxin Hc fragment of sequence SEQ ID NO 5, the Clostridium botulinum F serotype toxin Hc fragment of sequence SEQ ID NO 6, the Clostridium botulinum G serotype toxin Hc fragment of sequence SEQ ID NO 7, and the Clostridium tetani toxin Hc fragment of sequence SEQ ID NO 8, as well as variants thereof.

9. A method according to claim 1, wherein the genetic construct includes, at the 5′ end of the nucleic acid encoding at least one fragment of said toxin, the cytomegalovirus (CMV) promoter.

10. A method according to claim 1, wherein the genetic construct includes a sequence encoding an extracellular secretion signal.

11. A method according to claim 10, wherein the sequence encoding the extracellular secretion signal is selected from SEQ ID NO 9, which encodes for the mouse erythropoietin extracellular secretion signal, and SEQ ID NO 10, which encodes for the human alkaline phosphatase extracellular secretion signal, or a variant thereof.

12. A method according to claim 9, wherein the genetic construct includes at the 5′ end of the promoter a translation initiation site nucleic sequence, a so-called Kozak sequence, of sequence SEQ ID NO 11.

13. A method according to claim 1, wherein at least one initial codon of the nucleic acid sequence that encodes for at least one fragment of said toxin, is replaced by a different codon encoding the same amino acid and whose frequency in eukaryotic cells is greater than their frequency in Clostridium botulinum, as defined in Table 1.

14. A method according to claim 1, wherein the genetic construct also includes a nucleic acid encoding at least one cytokine.

15. A method according to claim 1, wherein the solution of step (a) includes another genetic construct that contains a nucleic acid encoding a cytokine, said two genetic constructs being co-administered in step (b).

16. A method according to claim 14 or 15, wherein the sequence of the nucleic acid encoding the cytokine is selected from the group consisting of SEQ ID NO 12, which encodes for the hematopoietic growth promoter (GM-CSF), SEQ ID NO 13, which encodes for mouse interleukin 12 subunit p35, SEQ ID NO 14, which encodes for mouse interleukin 12 subunit p40, SEQ ID NO 15, which encodes for mouse interleukin 4, and SEQ ID NO 16, which encodes for human interleukin 10.

17. A method according to claim 1, wherein the genetic construct also includes a non-methylated immunostimulation sequence rich in guanine and cytosine bases, between 10 and 10,000 nucleotides in size.

18. A method according to claim 1, wherein the antiserum is directed against at least two protein toxins and the solution in step (a) comprises a mixture of at least two genetic constructs, each of said constructs comprising a nucleic acid encoding at least one immunogenic fragment of said toxins.

19. A method according to claim 1, wherein the animal is selected from the group consisting of mice, rabbits, horses and pigs.

20. A method according to claim 1, wherein steps (b) and (c) are repeated at least once before step (d).

21. A method according to claim 1, wherein step (c) is followed by administering to the animal a recombinant immunogenic fragment of said toxin.

22. An antiserum directed against a protein toxin obtainable by the method according to claim 1, wherein the antiserum comprises antitoxin antibody titer equal to or greater than 100, and neutralizing strength equal to or greater than 100.

23. An antiserum according to claim 22, wherein the antiserum is administered as a preventative serum or antidote to neutralize in a mammal the toxic effects related to the absorption of the toxin in said mammal.

24. A method for preventing or treating a toxic effect related to absorption by a mammal of a toxin selected from the group consisting of Clostridium botulinum toxin, Clostridium tetani toxin, Bacillus anthracis toxin, ricin, diphtheria toxin and cholera toxin, comprising the administration by electrotransfer of an effective amount of a solution containing at least one genetic construct to a mammal in need thereof, wherein said genetic construct comprises a nucleic acid encoding at least one immunogenic fragment of the toxin.

25. A method according to claim 24, wherein the solution also contains an immunostimulator adjuvant.


SEQUENCE LISTING (17 sequences)

SEQ ID NO: 1 - protein, 427 aa, Clostridium botulinum
  C-terminal (Hc) fragment of toxin A
MENIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 432 aa, Clostridium botulinum
  C-terminal (Hc) fragment of toxin B
MLNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 3 - protein, 297 aa, Clostridium botulinum
  C-terminal (Hc) fragment of toxin C
NNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRV

SEQ ID NO: 4 - protein, 416 aa, Clostridium botulinum
  C-terminal (Hc) fragment of toxin D
FNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 5 - protein, 408 aa, Clostridium botulinum
  C-terminal (Hc) fragment of toxin E
MRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 6 - protein, 415 aa, Clostridium botulinum
  C-terminal (Hc) fragment of toxin F
GSIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 7 - protein, 432 aa, Clostridium botulinum
  C-terminal (Hc) fragment of toxin G
SSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 8 - protein, 451 aa, Clostridium tetani
  C-terminal (Hc) fragment of Clostridium tetani toxin
KNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYDTNEYSIISSMKKYSLSIGSGWSVSLKGNNLIWTLKDSAGEVRQITFRDLSDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVAYSSKDVQLKNITDYMYLTNAPSYTNGKLNIYYRRLYSGLKFIIKRYTPNNEIDSFVRSGDFIKLYVSYNNNEHIVGYPKDGNAFNNLDRILRVGYNAPGIPLYKKMEAVKLRDLKTYSVQLKLYDDKDASLGLVGTHNGQIGNDPNRDILIASNWYFNHLKDKTLTCDWYFVPTDEGWTND

SEQ ID NO: 9 - DNA, 87 bp, Mus musculus
  Extracellular secretion signal of mouse erythropoietin
atgggggtgcccgaacgtcccaccctgctgcttttactctccttgctactgattcctctgggcctcccagtcctctgtgctccccca

SEQ ID NO: 10 - DNA, 60 bp, Homo sapiens
  Extracellular secretion signal of human alkaline phosphatase
atgctgctgctgctgctgctgctgggcctgaggctacagctctccctgggcatcatccca

SEQ ID NO: 11 - DNA, 6 bp, Artificial Sequence
  Description of Artificial Sequence Synthetic kozac sequence
gccacc

SEQ ID NO: 12 - DNA, 916 bp, Mus musculus
  Colony stimulating factor (GM-CSF)
gagctcagcaagcgctctcccccaattcccttagccaaagtggacgccaccgaacagacagacctaggctaagaggtttgatgtctctggctacccgactttgaaaattttccgcaaaggaaggccttttgactacaatggcccacgagagaaaggctaaggtcctgaggaggatgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaatgaggaagcccaggccagctctgaatccagcttctcagactgctgcttttgtgcctgcgtaatgagccaggaactcggaatttctgccttaaagggaccaagagatgtggcacagccacagttggagggcagtatagccctctgaaaacgctgactcagcttggacagcggcaagacaaacgagagatattttctactgatagggaccattatatttatttatatatttatattttttaaatatttatttatttatttatttaattttgcaactctatttattgagaatgtcttaccagataataaattattaaaacttt

SEQ ID NO: 13 - DNA, 1281 bp, Mus musculus
  Sub-unit p35 of interleukin 12
tgccacctactcccttggatctgagctggacccttgcatctggcgtctacactgctgctgaaatcttctcaccgtgcacatccaaggatatctctatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctgaaaggctcaaggccctctgccacagcgccctcctcacacagataggaaacaaagaaagattcataagagtcaggtggtcttggcctggtgggccttaagctccttcaggaatctgttctcccatcacatctcatctccccaaaggtggcacagctacctcagcatggtcccctccatcgcttctctcatattcactatacaagttgtttgtaagttttcatcaaaatattgttaaggggcgaagacgtcctcccctcaatgtgttagcagaagagcaagaactgataagctattgtttttgtgccaaagtgtttatgaaaacactcagtcaccccttatttaaaaatatttattgctatattttatactcatgaaagtacatgagcctatttatatttatttattttctatttattataatatttcttatcagatgaatttgaaacattttgaaacataccttattttgtggttct

SEQ ID NO: 14 - DNA, 1008 bp, Mus musculus
  Sub-unit p40 of interleukin 12
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcgtgcagcaagtgggcatgtgttccctgcagggtccgatcctag

SEQ ID NO: 15 - DNA, 605 bp, Mus musculus
  Interleukin 4
ggatccccgggcagagctggggggggatttgttagcatctcttgataaacttaattgtctctcgtcactgacggcacagagctattgatgggtctcaacccccagctagttgtcatcctgctcttctttctcgaatgtaccaggagccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcgtagtactgagccaccatgctttaacttatgaatttttaatggttttattttaatatttatatatttataattcataaaataaaatatttgtataatgt

SEQ ID NO: 16 - DNA, 1629 bp, Homo sapiens
  Interleukin 10
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 17 - DNA, 1284 bp, Clostridium botulinum
  Hc fragment of toxin A
atggagaatattattaatacttctatattgaatttaagatatgaaagtaatcatttaatagacttatctaggtatgcatcaaaaataaatattggtagtaaagtaaattttgatccaatagataaaaatcaaattcaattatttaatttagaaagtagtaaaattgaggtaattttaaaaaatgctattgtatataatagtatgtatgaaaattttagtactagcttttggataagaattcctaagtattttaacagtataagtctaaataatgaatatacaataataaattgtatggaaaataattcaggatggaaagtatcacttaattatggtgaaataatctggactttacaggatactcaggaaataaaacaaagagtagtttttaaatacagtcaaatgattaatatatcagattatataaacagatggatttttgtaactatcactaataatagattaaataactctaaaatttatataaatggaagattaatagatcaaaaaccaatttcaaatttaggtaatattcatgctagtaataatataatgtttaaattagatggttgtagagatacacatagatatatttggataaaatattttaatctttttgataaggaattaaatgaaaaagaaatcaaagatttatatgataatcaatcaaattcaggtattttaaaagacttttggggtgattatttacaatatgataaaccatactatatgttaaatttatatgatccaaataaatatgtcgatgtaaataatgtaggtattagaggttatatgtatcttaaagggcctagaggtagcgtaatgactacaaacatttatttaaattcaagtttgtatagggggacaaaatttattataaaaaaatatgcttctggaaataaagataatattgttagaaataatgatcgtgtatatattaatgtagtagttaaaaataaagaatataggttagctactaatgcatcacaggcaggcgtagaaaaaatactaagtgcattagaaatacctgatgtaggaaatctaagtcaagtagtagtaatgaagtcaaaaaatgatcaaggaataacaaataaatgcaaaatgaatttacaagataataatgggaatgatataggctttataggatttcatcagtttaataatatagctaaactagtagcaagtaattggtataatagacaaatagaaagatctagtaggactttgggttgctcatgggaatttattcctgtagatgatggatggggagaaaggccactgtaa